(19) United States
(12) Patent Application Publication
(10) Pub. No.: US 2014/0370129 A1
(43) Pub. Date: Dec. 18, 2014

(54) DOSING REGIMEN AND METHOD FOR TREATING CANCER USING A COIX SEED OIL EMULSION

(71) Applicant: Zhejiang Kanglaite Group Co., Ltd., Hangzhou (CN)

(72) Inventor: Dapeng Li, Hangzhou (CN)

(21) Appl. No.: 13/918,121

(22) Filed: Jun. 14, 2013

(51) Int. Cl. A61K 36/8994

(52) U.S. Cl. CPC A61K 36/8994
USPC 424/750

(57) ABSTRACT

A method for administering a Kanglaite Injection (KLTi) composition comprising coix seed oil to a subject diagnosed with cancer is described. The method comprises administering daily doses of the KLTi on consecutive days for at least a first and second time period with a rest period between the first and second time periods during which no KLTi is administered.

### CROSS-REFERENCE TO RELATED APPLICATIONS

This field is intentionally left blank.

### TECHNICAL FIELD

The subject matter described herein relates to the use of an emulsion of an oil extracted from seeds of Coix lacryma-jobi, referred to as Kanglaite Injection (KLTi), for the prevention, ameloriation and/or treatment of a cancer, including solid tumors, and in particular to a dosing regimen and methods of treatment using KLTi.

### BACKGROUND

Pancreatic cancer is the fourth leading cause of cancer-related death in the United States, and is second to colorectal cancer as a cause of digestive cancer-related death (Jemal et al., 2006, CA Cancer J Clin, 56:106-130; NCCN Clinical Practice Guideline in Oncology, Pancreatic Adenocarcinoma, 2006, v. 2). The incidence of pancreatic cancer in the U.S. has been increasing since the 1930s, but has remained relatively stable since the 1970s. Tumors of the exocrine pancreas account for over 90% of pancreatic cancers, with ductal adenocarcinoma being the predominant histological diagnosis, affecting more than 30,000 people each year in the US (Li et al., 2004, Lancet, 363:1049-1057; Eckle et al., 2006, Expert Opin Investig Drugs, 15:1395-1410). Mortality rates closely parallel incidence rates because of poor prognosis (NCCN Clinical Practice Guideline in Oncology, Pancreatic Adenocarcinoma, 2006, v. 2, Eckle et al., 2006, Expert Opin Investig Drugs, 15:1395-1410). The rate of cure in all patients is <1% (Eckle et al., 2006, Expert Opin Investig Drugs, 15:1395-1410). The incidence is higher in men and blacks, and it is rare before age 45, rising sharply thereafter. Known risk factors are age, smoking, history of chronic pancreatitis, diabetes mellitus, and hereditary/genetic predisposition to pancreatic cancer specifically or to cancer in general (Jemal et al., 2006, CA Cancer J Clin, 56:106-130; Li et al., 2004, Lancet, 363:1049-1057).

Current therapeutic options for treating patients diagnosed with pancreatic cancer include surgery, radiation therapy, chemotherapy and chemoradiation therapy. The method of treatment used is generally determined by the stage of the pancreatic cancer. If imaging tests show a reasonable chance of completely removing the cancer, surgery will likely be performed. However, even if surgery has removed all of the tumor that is visible, the cancer often comes back. Studies have shown that giving chemotherapy after surgery can delay the cancer's return by about 6 months, such as by treatment with gemcitabine or 5-fluorouracil. Some patients are given chemotherapy alone or with radiation therapy before surgery. Chemotherapy and/or radiation is also the treatment most commonly used for locally advanced tumors and metastatic cancer.

A standard treatment for advanced pancreatic cancer is chemotherapy using gemcitabine which can shrink the cancer and help patients live longer. Adding other drugs to gemcitabine has shown to improve the chance the tumors will shrink and prolong life of the patient. So far, only erlotinib and capecitabine have been shown to be most beneficial in combination with gemcitabine, though recent results suggest that a combination of ABRAXANE (paclitaxel protein-bound particles) with gemcitabine may also provide therapeutic efficacy in the treatment of pancreatic cancer. Another option that may help patients live longer is a combination of chemotherapeutic drugs called FOLFIRINOX which consists of 5-fluorouracil, leucovorin, irinotecan and oxaliplatin. While studies have shown greater efficacy as compared to gemcitabine, patients treated with FOLFIRINOX also experienced greater side effects.

Accordingly, there remains a significant need for improved methods and compositions for treating cancers, including pancreatic cancer.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a dosing regimen is provided, comprising administering intravenously a first dose of an emulsion of an oil extracted from seeds of Coix lacryma-jobi (KLTi) each day for a first dosing time period of 3 to 7 consecutive days, waiting for a first resting time period of 1 to 5 days wherein during the resting time period KLTi is not administered, and repeating the administering at least once at a second dose of KLTi each day for a second dosing time period of 3 to 7 consecutive days. In one embodiment, the dosing regimen is provided to a patient diagnosed with cancer. In another embodiment, the patient is undergoing treatment with a chemotherapeutic agent.

In one embodiment, the first dose of KLTi comprises about 20 grams (g) to 50 g coix seed oil. In another embodiment, the first dose of KLTi comprises about 30 g to 40 g coix seed oil. In still another embodiment, the first dose of KLTi comprises about 20 g, 30 g, 40 g, or 50 g coix seed oil.

In one embodiment, the concentration of coix seed oil in each dose of KLTi is 5 g/100 mL to 25 g/100 mL. In another embodiment, the concentration of coix seed oil is 10 g/100 mL.

In one embodiment, the first dosing time period is 4 to 5 consecutive days. In another embodiment the first dosing period is 3, 4, 5, 6, or 7 consecutive days. In still another embodiment, the first dosing period is 5 consecutive days.

In one embodiment, the first resting time period is 2 to 4 consecutive days. In still another embodiment, the first resting period is 1, 2, 3, 4, or 5 consecutive days. In yet another embodiment, the first resting period is 2 consecutive days.

In one embodiment, the second dose of KLTi comprises about 20 g to 50 g coix seed oil. In another embodiment, the second dose of KLTi comprises about 30 g to 40 g coix seed oil. In still another embodiment, the second dose of KLTi comprises about 20 g, 30 g, 40 g, or 50 g coix seed oil.

In one embodiment, the second dosing time period is 4 to 5 consecutive days. In another embodiment the second dosing period is 3, 4, 5, 6, or 7 consecutive days. In still another embodiment, the second dosing period is 5 consecutive days.

In one embodiment, the regimen further comprises waiting for a second resting time period of between 1 to 5 days, wherein during the second resting time period KLTi is not administered. In another embodiment, the second resting time period is between 2 to 4 days. In still another embodiment, the second resting period is 1, 2, 3, 4, or 5 days. In yet another embodiment, the second resting period is 2 consecutive days.

In one embodiment, the regimen further comprises administering a third dose of KTLi for a third dosing time period of between 3 to 7 consecutive days. In another embodiment, the third dosing time period is 4 to 5 consecutive days. In another embodiment the third dosing period is 3, 4, 5, 6, or 7 consecutive days. In still another embodiment, the third dosing period is 5 consecutive days.

In one embodiment, the third dose of KLTi comprises about 20 g to 50 g coix seed oil. In another embodiment, the third dose of KLTi comprises about 30 g to 40 g coix seed oil. In still another embodiment, the third dose of KLTi comprises about 20 g, 30 g, 40 g, or 50 g coix seed oil.

In one embodiment, the regimen comprises waiting for a third resting period of 3 to 11 consecutive days. In another embodiment the third resting period is 4 and 10 consecutive days. In yet another embodiment, the third resting period is 3, 4, 5, 6, 7, 8, 9, 10 or 11 consecutive days.

In one embodiment, the regimen of treatment occurs over a period of 25 to 35 days. In another embodiment, the regimen of treatment occurs over a period of 25, 26, 27, 28, 29 or 30 days. In yet another embodiment, the regimen of treatment is repeated at least 2, 3, 4, 5, 6, 7, 8, or 9 times.

In one embodiment, the number of first, second and third dosing time period days and the first, second and third resting period days combine to a total treatment cycle having 28 days.

In one embodiment, the first dose of KLTi is administered for a first dosing time period of between 4 to 6 consecutive days. In another embodiment, the first dose of KLTi is administered for a first dosing time period of 4, 5, 6 or 7 consecutive days.

In one embodiment, the subject has been diagnosed with a cancer selected from the group consisting of pancreatic, breast, gastric, non small cell lung carcinoma (NSCLC) or liver cancer.

In one embodiment the subject has been diagnosed with a Stage 1, Stage 2, Stage 3, or Stage 4 cancer. In another embodiment, the subject has been diagnosed with a metastatic cancer.

In one embodiment, the cancer is non-resectable.

In one embodiment, the subject is being and/or has been treated with at least one chemotherapeutic agent. In another embodiment, the chemotherapeutic agent is selected from the group consisting of fluorouracil, erlotinib hydrochloride, gemcitabine hydrochloride, ABRAXANE, mitomycin-C, or any combination thereof. In one embodiment, the subject is being and/or has been treated with FOLFIRINOX. In another embodiment, the subject is being and/or has been treated with ABRAXANE and gemicitabine hydrochloride.

In one embodiment, the subject is being and/or has been treated with radiation therapy.

In another aspect, a method of treating cancer is provided, wherein KLTi is administered in accord with a dosing regimen set forth herein.

In one embodiment, the method comprises administering to a subject intravenously a first dose of KLTi each day for a first dosing time period of 3 to 7 consecutive days, waiting for a first resting time period of 1 to 5 days wherein during the resting time period KLTi is not administered, and repeating the administering at least once at a second dose for a second dosing time period of 3 to 7 consecutive days, wherein the subject has been diagnosed with a cancer.

In one embodiment, the first dose of KLTi comprises about 20 g/day to 50 g/day coix seed oil.

In one embodiment, the first dosing time period is 4 to 5 consecutive days.

In one embodiment, the first resting time period is 2 to 4 consecutive days.

In one embodiment, the second dose of KLTi comprises about 20 g/day to 50 g/day coix seed oil.

In one embodiment, the second dosing time period is 4 to 5 consecutive days.

In one embodiment, the method further comprises waiting for a second resting time period of 1 to 5 days, wherein during the second resting time period KLTi is not administered.

In one embodiment, the method further comprises administering a third dose of KTLi for a third dosing time period of 3 to 7 consecutive days.

In one embodiment, the third dose of KLTi comprises about 20 g/day to 50 g/day coix seed oil.

In one embodiment, the method further comprises waiting for a third resting period of 3 to 11 consecutive days.

In one embodiment, the subject has been diagnosed with a cancer.

In one embodiment, the cancer is pancreatic, prostate, non small cell lung carcinoma, gastric, breast or liver cancer.

In one embodiment, the subject is being treating with a chemotherapeutic agent.

In one embodiment, the subject is being treated with a chemotherapeutic agent selected from the group consisting of fluorouracil, erlotinib hydrochloride, gemcitabine hydrochloride, mitomycin-C, ABRAXANE and FOLFIRINOX.

In another aspect, a dosing regimen is provided, comprising directing a composition of KTLi to be administered to a subject during a first dosing time period wherein the KTLi provides coix seed oil at a dose of 20 g/day to 50 g/day for 3 to 7 consecutive days, the first dosing time period followed by a first resting time period during which the composition is not administered, and repeating at least once.

In one embodiment, the repeating at least once consists of administering to the subject during a second dosing time period the composition of KTLi wherein the KTLi provides coix seed oil at a dose of 20 g/day to 50 g/day for 3 to 7 consecutive days, the second dosing time period followed by a second resting time period during which the composition is not administered.

In one embodiment, the dosing regimen further comprises directing the composition of KTLi to be administered to the subject during a third dosing time period wherein the KTLi provides a third dose of coix seed oil at a dose of 20 g/day to 50 g/day for 3 to 7 consecutive days, the third dosing time period followed by a third resting time period during which the composition is not administered.

In one embodiment, the first resting time period is 2 to 4 consecutive days.

In one embodiment, the second resting time period is 2 to 4 consecutive days.

In one embodiment, the third resting time period is 3 to 11 consecutive days.

In one embodiment, the third dose of KLTi comprises about 20 g/day to 50 g/day coix seed oil.

In one embodiment, the subject has been diagnosed with a cancer.

In one embodiment, the subject has been diagnosed with a cancer selected from the group consisting of pancreatic, prostate, non small cell lung carcinoma, gastric, breast or liver cancer.

In one embodiment, the subject is being treating with a chemotherapeutic agent.

In one embodiment, the subject is being treated with a chemotherapeutic agent selected from the group consisting of fluorouracil, erlotinib hydrochloride, gemcitabine hydrochloride, mitomycin C, and FOLFIRINOX.

In another aspect, a kit is provided, comprising at least one intravenous bag containing KLTi. In one embodiment, the kit further comprises a chemotherapeutic agent.

Additional embodiments of the present methods and treatment regiments will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF DRAWINGS

FIG. 1 (Product-Limit Survival Estimates for Overall Survival (in Days)) shows the survival probability, based on overall survival analysis of patients treated with KLTi plus gemcitabine (solid line) or with gemcitabine alone (dashed line).

FIG. 2 (Product-Limit Survival Estimates for Progression-Free Survival (in Days)) shows the survival probability based on PFS analysis of for patients treated with KLTi plus gemcitabine (solid line) or with gemcitabine alone (dashed line).

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### I. DEFINITIONS

As used in this specification, the singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The term “administration” or “administering” includes routes of introducing the compound of the invention(s) to a subject to perform their intended function.

The term “effective amount” or “therapeutically effective amount,” includes an amount effective, at dosages and for periods of time necessary, to achieve the desired result, e.g., sufficient to treat a cell proliferative disorder. An effective amount of composition as described herein may vary according to factors such as the disease state, age, and weight of the subject, and the ability of the compound of the invention to elicit a desired response in the subject. Dosage regimens may be adjusted to provide the optimum therapeutic response. An effective amount is also one in which any toxic or detrimental effects (e.g., side effects) of the compound of the invention are outweighed by the therapeutically beneficial effects. A therapeutically effective amount of a composition refers to an amount of the composition of or an agent which is effective, upon single or multiple dose administration to the patient, in inhibiting cell proliferation and/or symptoms of a cell proliferative disorder, or in prolonging the survivability of the patient with such a cell proliferative disorder beyond that expected in the absence of such treatment.

The term “subject” includes organisms which are capable of suffering from a cell proliferative disorder or who could otherwise benefit from the administration of a compound of the invention of the invention, such as human and non-human animals. Preferred humans include human patients suffering from or prone to suffering from a cell proliferative disorder or associated state, as described herein. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc.

The phrases “systemic administration,” “administered systemically”, “peripheral administration” and “administered peripherally” as used herein mean the administration of a compound of the invention(s), drug or other material, such that it enters the patient's system and, thus, is subject to metabolism and other like processes, for example, subcutaneous administration.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include pancreatic cancer, lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, squamous cell cancer (e.g. epithelial squamous cell cancer), thyroid cancer, hepatic carcinoma, anal carcinoma, penile carcinoma, as well as head and neck cancer.

The term “emulsion,” as used herein refers to a mixture of two or more immiscible liquids. In an emulsion, at least one of the liquids is present as droplets that are dispersed throughout the other liquid(s), which form a continuous phase. For example, an oil-in-water emulsion is a mixture of oil and water in which the oil is present as droplets dispersed throughout the water.

### II. METHODS OF TREATMENT AND DOSING REGIMENS

The present disclosure is directed to the discovery and development of a dosing regimen using Kanglaite Injection (KLTi) comprising coix seed oil which has shown to be surprisingly efficacious for the treatment of pancreatic cancer when combined with standard treatment with gemcitabine. As described in more detail below, studies both in vivo and in vitro have shown that KLTi can slow the growth of cancerous cells and suggest that KLTi has therapeutic value for the treatment of numerous types of cancer.

Kanglaite Injection (KLTi) is a botanical product that was approved in China in 1995 for the treatment of non-small cell lung cancer (NSCLC) and hepatic cancer. Additionally, it is used in the treatment of cachexia due to cancer, cancer-related pain, and other systemic manifestations of the disease. The active ingredient of Kanglaite Injection is a neutral oil extracted and isolate from the seed of the coix plant, Coix lacryma-jobi, which is a member of the grass family and is also known as Job's tears. The extraction process yields purified coix seed oil (neutral lipids from the endospesrm of Job's tears or NLEJ) which is formulated as a micro-emulsion for intravenous administration. The coix seed oil emulsion (KLTi) as used herein was prepared by the Zhejiang Kanglaite Pharmaceutical Co., using seeds harvested from specified fields in the Zhejiang Province of China. The preparation method is described in detail in Example 1.

Coix seed oil is by weight composed of over 90% triglycerides with smaller amounts of diglycerides (about 1.5%), monoglycerides (about 6%) and alkylacylacetin (about 1%), and its three main active ingredients are palmitic acid, oleic acid, and linoleic acid. The composition of the KLTi active ingredient as used herein is: 91.5% triglycerides, 5.8% monoglyceride, 1.5% diglyceride, and 1.0% alkyl fatty acid. C-18 unsaturated and C-16 saturated fatty acids account for 96% of the triglyceride component. It is readily envisioned that each of these percent values listed for the active ingredient used in the therapeutic methods presently embodied may vary by about ±0.5%, ±1%, ±5%, or ±10%.

Kanglaite Injection which is supplied from China is provided in 100 mL glass bottles compatible with standard intravenous infusion sets. Any Kanglaite Injection emulsion prepared for use in therapeutic methods as described herein may be prepared and provided in containers suitable for storage, delivery and/or administration to a subject. The emulsion prepared for use as described herein for therapeutic treatment of subjects contains 10 g coix seed oil per 100 mL of emulsion. For the purpose of the described methods, pharmaceutical formulations may be used which contain higher or lower concentrations of coix seed oil, for example but not limited to, 1 g/100 mL to 50 g/100 mL, 5 g/100 mL to 25 g/100 mL, 5 g/100 mL to 40 g/100 mL, 10 g/100 mL to 20 g/100 mL, or 5 g/100 mL to 15 g/100 mL.

A method for the extraction and refinement of these neutral lipids from the endosperm of Job's tears (NLEJ) is described in detail in U.S. Pat. No. 5,444,089, incorporated by referenced herein in its entirety. The method of emulsification includes two conventional steps, homogenization and dispersion.

To summarize, the extraction is performed using an organic solvent such as acetone, petrol ether, ether, alcohol or hexane. The extraction method may be percolation, filtering or seeping through the powder of endosperm of Job's tears. Crude oil will be extracted with large amounts of foreign matter such as free fatty acids and pigments. A suitable amount of alkaline solution (e.g., NaOH or KOH) is added for alkalizing saponification, resulting in an emulsion. Deemulsification by a suitable amount of acetone will cause the emulsion to become transparent. Then a suitable amount of a second organic solvent, e.g., petrol ether, either or hexane, is used. The liquid is extracted and allowed to settle, the phase containing mainly acetone (containing acidic element and water) is discarded, and the organic solvent is evaporated, leaving a residue comprising mainly NLEJ.

Between the steps of alkalization saponification and deemulsification with acetone, a step of washing the emulsion with hot water may be added as a means of refinement. And/or after the liquid-liquid extraction step, one step of decoloration and/or washing by hot water may be added if necessary. Then NLEJ of high purity meeting the need of intravenous use is obtained.

The emulsion as a pharmaceutical composition contains NLEJ as the main component, soy (or egg) lecithin as an emulsifier and enough water. It also contains glycerol, sorbitol or the like as an isoosmoticum. It may also contain other conventional anti-tumor drugs.

In some embodiments, the neutral lipid isolated from the endosperm of Job's tears (NLEJ; coix seed oil) consists essentially of glycerides and alkylacylacetin, wherein the lipoclastic fatty acid residues of the coix seed oil comprise saturated and unsaturated fatty acids, and wherein the neutral lipid has the following physicochemical properties: acid value <0.20, iodine value 95.00-107.00, saponification value 185.00-195.00, relative density 0.915-0.918 (20° C.).

Coix seed oil as used here may also be extracted from coix seed using methods detailed in Chinese patent no. CN1200995C (“Method for extracting coarse oil from coix seed with hypercritical carbon dioxide”). Similarly, the following method may be used to extract coix seed oil: coix seeds are dried until their water content is below 10%. The seeds are then ground to fine particles. The fine particles are further extracted in the extraction vessel of a supercritical extraction system under the following conditions: in the extracting vessel, the temperature is about 30° C. to about 60° C., the pressure is about 19 MPa to about 24 MPa. In the separating column, the temperature is about 30° C. to about 60° C., and the pressure is about 6 MPa to about 15 MPa. In the resolving vessel the temperature is about 30° C. to about 60° C., the pressure is about 2 MPa to about 6 MPa and the flow rate is 10-5000 L/hour.

In some embodiments, the emulsion of coix seed oil is prepared for intravenous use by combining 10 g coix seed oil, 1.5 g soy lecithin, and 2.5 g glycerol, adding water for Injection until the volume of the mixture is 100 mL, and using the combined ingredients to make the emulsion. As an alternative, the emulsion of coix seed oil is prepared for intravenous used by combining 15 g coix seed oil, 2.0 g soy lecithin, and 2.5 g glycerol, adding water for Injection until the volume of the mixture is 100 mL, and using the combined ingredients to make the emulsion.

### III. PANCREATIC CANCER AND TREATMENT THEREOF

The methods of treatment described herein are based on the development of new dosing regimens for the intravenous administration of KLTi which have proven in the clinic to be therapeutically effective in slowing the progression of pancreatic cancer in patients diagnosed as having locally advanced or metastatic pancreatic cancer, not amenable to curative surgical resection. However, the skilled artisan will appreciate that the methods for administering KLTi described in more detail below may be used to treat patients diagnosed with any stage or type of pancreatic cancer. These different types and stages of pancreatic cancer are described below.

There are several types of pancreatic cancer which are classified in part on whether the cancer began in the exocrine or endocrine component. The most common type of pancreatic cancer exocrine tumors, adenocarcinomas which begin in glandular cells. These tumors usually originate in the ducts of the pancreas to form ductal adenocarcinomas. If the tumor begins in the acini, it is called acinar adenocarcinoma. An intraductal papillary mucinous neoplasm (IPMN) refers to a tumor that grows within the ducts of the pancreas and makes a thick fluid called mucin. IPMN is not cancerous when it begins, but could become cancerous if not treated. Rarer types of exocrine pancreatic tumors include acinal cell carcinoma, adenosquamous carcinoma, colloid carcinoma, giant cell tumor, hepatoid carcinoma, mucinous cystic neoplasms, pancreatoblastoma, serous cystdenoma, signet ring cell carcinoma, solid and pseudopapillary tumors, squamous cell carcinoma, and undifferentiated carcinoma.

Pancreatic tumors of endocrine origin are also called islet cell tumors or pancreatic neuroendocrine tumors (PNETs). They are much less common than exocrine tumors, making up about 1% of pancreatic cancers. A pancreatic neuroendocrine tumor can be functioning, meaning it makes hormones, or nonfunctioning, meaning it doesn't make hormones. A functioning neuroendocrine tumor is named based on the hormone the cells normally make, and include, insulinoma, glucagonoma, gastrinoma, somatostatinoma, VIPomas and PPomas.

The KLTi treatment regimens described herein may be used for patients diagnosed with any of the various stages of cancer. These stages include: Stage 0 in which the pancreatic cancer is limited to a single layer of cells in the pancreas (the cancer is not visible in imaging tests or to the naked eye); Stage I: Local growth in which the cancer is limited to the pancreas, but has grown to less than 2 centimeters across (stage IA) or greater than 2 centimeters (stage IB); Stage II: Local spread of the cancer in which the cancer has grown outside the pancrease and/or has spread to nearby lymph nodes; Stage III: Wider spread in which the tumor has expanded into nearby major blood vessels or nerves but has not metastasized; and Stage IV: Confirmed spread in which the pancreatic cancer has spread to distant organs. Staging of cancer is may be done through imagine tests such as CT scans and ultrasound.

Upon diagnosis of pancreatic cancer, a doctor will determine whether the cancer appears to be removable by surgery (is resectable). Pancreatic cancer is considered resectable if the tumor appears to be localized to the pancreas without invasion into important surrounding structures such as the mesenteric blood vessels which are located adjacent to the head portion of the pancreas. In some embodiments a tumor is considered resectable if there is no evidence of metastatic spread to the liver or lining of the intestines. Two operations commonly performed for removal of a pancreatic tumor include the Whipple operation and distal pancreatectomy and splenectomy. Methods of treatment disclosed herein may be performed in subjects having resectable or non-resectable cancer.

For chemotherapeutic treatment of pancreatic cancer, doctors commonly use gemcitabine and fluorouracil. Other drugs that may be combined with gemcitabine or fluorouracil include capecitabine, cisplatin, docetaxel, irinotecan, oxaliplatin and paclitaxel. For pancreatic neuroendocrine tumors, doctors commonly prescribe doxorubicin, streptozocin, liposomal doxorubicin, fluorouracil, dacarbazine and/or temozolomide. Accordingly, KLTi administration may be useful in combination with treatment of one or more of the above listed chemotherapeutic agents, based on the cancer being treated.

### IV. METHODS OF ADMINISTRATION

KLTi may be administered, for example, by intravenous or intra-arterial infusion. Dosages may be administered as part of a dosage regimen as described in more detail below.

As shown through clinical studies, described in Example 2, intravenous administration of KLTi is significantly effective in treating pancreatic cancer when administered in combination with gemcitabine to a patient diagnosed with pancreatic cancer.

Standard treatment with gemcitabine involves dosing based on body surface area (BSA) of the patient. Gemcitabine is administered via intravenous infusion over a 30-minute time period at one-week intervals, in order to provide a rest period. KLTi is also administered via intravenous infusion on two or more consecutive days with rest periods in between the period of consecutive days.

Based on the clinical studies described herein, dosing regimens have been identified in which KLTi is administered via intravenous infusion such that about 20 g to 50 g of coix seed oil in the pharmaceutical KLTi composition is infused over a time period. The time needed to infuse a dose of KLTi depends upon the concentration of the coix seed oil emulsion and the rate at which the infusion can occur. When determining a rate of infusion, the effects of time on patient compliance, to avoid excessive infusion times, can be considered. The rate of infusion may be varied during the administration of a dose of KLTi. For example, the infusion rate may be started at about 1.0 mL/min for the first 15 minutes then increased to 150 mL/hour until the KLTi is completely infused. A target rate of infusion may be 200 mL/hour (3.33 mL/min). Accordingly, the infusion rate may vary from, from example, about 0.5 mL/min to 5 mL/min or from about 1 mL/min to 3.3 mL/min.

It is envisioned that any pharmaceutically acceptable coix seed oil (NLEJ) emulsion which can provide infusion of 30 g to 50 g of the coix seed oil to the patient within a time period of 2 to 4 hours, 1 to 3 hours, or 2 to 3 hours will be effective as well as convenient for the patient.

An advantage of the present methods is the ability to administer the KLTi via a portable infusion pump operated in the patient's home.

In one embodiment, a patient which has been diagnosed with pancreatic cancer and who is undergoing treatment with gemcitabine is treated with KLTi according to a dosing regimen which is designed as a 28-day cycle, which may or may not be repeated. The regimen includes administering KLTi to the patient as an intravenous infusion. A first dose of KLTi is administered on 5 consecutive days (i.e., Days 1, 2, 3, 4, and 5). The patient is then allowed a period of rest during which no KLTi is administered for 2 days (i.e., Days 6 and 7). The patient is then administered a dose of KLTi an additional 5 consecutive days (i.e., Days 8, 9, 10, 11, and 12) then allowed to rest for another 2 days (no KLTi is administered on Days 13 and 14). KLTi is then administered to the patient for another 5 consecutive days (i.e., Days 15, 16, 17, 18, and 19) and then allowed to rest for 9 consecutive days (i.e., Days 20, 21, 22, 23, 24, 25, 26 and 28).

During this 28-day cycle, the patient is also receiving standard gemcitabine treatment. This involves intravenous administration of gemcitabine at a dose of 1000 mg/m² on Days 1, 8 and 15. However, KLTi administration as described above would be useful in combination with any prescribed dosing regimen for gemcitabine.

The 28-day cycle is repeated for as long as the patient is willing to comply and is healthy enough to receive the treatment.

One having ordinary skill in the art would envision that the efficacy observed when treating a subject having pancreatic cancer with the above-described dosing regimen would likely also be observed when using the same or similar dosing regimen for treating patients diagnosed with all stages of pancreatic cancer as well as patients diagnosed with other cancers such as NSCLC, gastric cancer, hepatocellular cancer and prostate cancer as described in more detail below.

The methods and dosing regimen described herein also envisions using the above-described dosing regimen for administering KLTi in the absence of gemcitabine administration, or instead, in the presence of another chemotherapeutic such as fluorouracil (5-FU), FOLFIRINOX, ABRAXENE, erlotinib or capecitabine. Moreover, there may be variations in the specified dosing regimen. For example, within a 16 to 46-day cycle, a first dose of KLTi may be administered daily for 3 to 7 consecutive days, followed by a first resting time period of 1 to 5 days, followed by a second dose of KLTi administered daily for 3 to 7 days, followed by a second resting time period of 1 to 5 days, followed by a third dose of KLTi administered daily for 3 to 7 days, followed by a third resting period of 5 to 15 days, or any combination of the above time period ranges.

In an exemplary embodiment, the KLTi composition which is administered to the patient contains approximately 10 g/100 mL coix seed oil. The first, second and third doses of KLTi composition contain approximately 30 g coix seed oil. However, the treatment regimen disclosed herein may effectively treat cancer when administered a pharmaceutically effective emulsion which contains coix seed oil at a concentration of 1 g/100 mL to 25 g/100 mL and wherein administration each of the first, second and third doses of KLTi composition delivers 20 g to 60 g coix seed oil, or 30 g to 50 g coix seed oil.

In one aspect, the disclosure encompasses intravenous bags containing KLTi suitable for intravenous administration to a subject. The IV bags containing the KLTi are suitable and convenient for home infusion, thus reducing the number of hospital visits, the length of hospital stays and the cost of medical care. The IV bags containing the KLTi emulsion of coix seed oil is made of ethylene-vinyl acetate (EVA) or polyolefin. In one embodiment, the IV bag is a Wold-Pak™ 1000 mL EVA TPN 3-Leg Gravity Bag, a Baxter 1000 mL Empty All-in-One EVA container for gravity transfer, or a Hospira VisIV™ container. The bag may be stored at a temperature between 2° C. and 25° C.

### VI. USE OF KANGLAITE INJECTION FOR TREATMENT OF OTHER CANCERS

KLTi is approved in China for treatment of patients with advanced NSCLC and advanced hepatocellular cancer and in Russia for treatment of patients with advanced NSCLC. In China and Russia, when used to treat advanced NSCLC, KLTi is typically used in conjunction with conventional chemotherapy and/or radiation therapy. It is, therefore, an adjunct to therapy, and is commonly used off-label as an adjunct to chemotherapy to treat patients with other solid tumors, such as pancreatic or esophageal cancer. Additionally, the drug is used alone for palliation of patients with advanced cancer of all types.

One having ordinary skill in the art can envision that the above dosing regimen for administering KLTi may be useful in treating cancers other than pancreatic cancer, including NSCLC, liver (hepatocellular), prostate, and gastric cancer. KLTi is approved in China for treatment of patients with advanced NSCLC and advanced hepatocellular cancer and in Russia for treatment of patients with advanced NSCLC. Based on a study published by Zhan et al (2012; Asian Pac J Cancer Prevention; 12:5319-5321) the authors concluded that KLTi enhanced efficacy and reduced the side effects of chemotherapy and improved the quality of life of gastric cancer patients. Patients in the experimental received KLTi in combination with the DOC regimen which consisted of docetaxel, oxaliplatin and capecitabine, while patients in the control group received the DOC treatment regimen only. KLTi may also be used as described herein for treatment of prostate cancer. As shown in U.S. Pat. No. 8,299,121 (incorporated herein by reference in its entirety), coix seed oil, when administered as KLTi or as a softgel, was effective in inhibiting the growth of human prostate cancer cells in a mouse model. Similarly, as described in the same U.S. patent, coix seed oil prepared as a softgel and administered with lupron was able to inhibit PC-3M tumor growth in nude mice which harbored these tumors.

### VII. COMBINATION THERAPIES

Drugs currently approved by the FDA for treating pancreatic cancer include fluorouracil (5-FU), erlotinib hydrochloride, gemcitabine hydrochloride, and mitomycin C. It is envisioned that administration of KLTi in combination with any one or more of the treatments described here would be efficacious in slowing the progression of cancer, including but not limited to, pancreatic, NSCLC, prostate, hepatic, or gastric cancer.

Gemcitabine (e.g., Gemzar®) has been a standard treatment for pancreatic cancer since the 1990s. Gemcitabine has demonstrated activity in patients with pancreatic cancer and is a useful palliative agent (Rothenberg et al., 1996, Ann Oncol, 7:347-353; Burris et al, 1997, J Clin Oncol, 15:2403-2413; Storniolo et al., Cancer, 85:1261-1268). A phase 3 trial of gemcitabine versus 5-FU as first-line therapy in patients with advanced or metastatic adenocarcinoma of the pancreas reported a significant improvement in survival among patients treated with gemcitabine (1-year survival was 18% with gemcitabine as compared with 2% with 5-FU, P=0.003 (Burris et al, 1997, J Clin Oncol, 15:2403-2413)). The National Cancer Institute of Canada performed a phase 3 trial that compared gemcitabine alone versus the combination of gemcitabine and erlotinib (100 mg/day) in patients with advanced or metastatic pancreatic carcinomas. This clinical study showed that the addition of erlotinib modestly prolonged survival when combined with gemcitabine alone (hazard ratio [HR], 0.81; 95% confidence interval [CI], P=0.038 (Storniolo et al., Cancer, 85:1261-1268). The median and 1-year survival rates for patients who received erlotinib versus placebo were 6.2 months and 5.9 months, and 23% versus 17%, respectively (Moore et al., 2007, J Clin Oncol, 25:1960-1966).

The approved dosing of gemcitabine for treatment of pancreatic cancer is 1000 mg/m² as an intravenous infusion over 30 minutes once weekly for the first 7 weeks, then 1 week rest, followed by once weekly administration for 3 weeks of 28-day cycles.

Gemcitabine is also approved by the FDA in combination with carboplatin for the treatment of advanced ovarian cancer that has relapsed at least 6 months after completion of platinum-based therapy, in combination with paclitaxel for first-line treatment of metastatic breast cancer after failure of prior anthracycline-containing adjuvant chemotherapy, unless anthracyclines were clinically contraindication, and in combination with cisplatin for the treatment of NSCLC. Accordingly, it is envisioned that each of the above-described uses of gemcitabine may be augmented by the further administration of KLTi as described herein. In other words, approved dosing regimens of gemcitabine for treatment of metastatic breast cancer, advanced ovarian cancer or NSCLC may be administered to a patient in need thereof in combination with intravenous infusion of KLTi at a dose of 20-50 g/day for a first time period of between 3-7 consecutive days, followed by a waiting period of between 1-5 days, which is then followed by administering a second dose of KLTi for a second time period of between 3-7 consecutive days, within a repeated 28-day cycle.

FOLFIRINOX is another treatment for pancreatic cancer which has been shown to be more efficacious than treatment with gemcitabine (Conroy et al., 2011, N Engl J Med, 364:1817-1825). FOLFIRINOX refers to a combination of 85 mg/m² oxaliplatin, 180 mg/m² irinotecan, 400 mg/m² leucovorin and 400 mg/m² fluorouracil given as a bolus followed by 2400 mg/m² given as a 46-hour continuous infusion every 2 weeks. A study was completed in which 342 patients with metastatic pancreatic adenocarcinoma that had not previously been treated with chemotherapy were divided and treated such that one group was received the FOLFIRINOX treatment while the second group received gemcitabine at a dose of 1000 mg/m² weekly for 7 of 8 weeks than then weekly for 3 of 4 weeks. The median overall survival was 11.1 months in the FOLFIRINOX group as compared with 6.8 months in the gemcitabine group. Accordingly, in one embodiment, subjects diagnosed with pancreatic cancer, may receive the FOLFIRINOX dosing regimen as described above, in combination with intravenous infusion of KLTi at a dose of 20-50 g/day for a first time period of between 3-7 consecutive days, followed by a waiting period of between 1-5 days, which is then followed by administering a second dose of KLTi for a second time period of between 3-7 consecutive days, within a repeated 28-day cycle.

Another promising therapeutic agent for treating pancreatic cancer is ABRAXANE (paclitaxel bound to albumin). ABRAXANE is presently FDA-approved for treatment of metastatic breast cancer after failure of combination chemotherapy for metastatic disease or relapse within 6 months of adjuvant chemotherapy, and for locally advanced or metastatic NSCLC, as first-line treatment in combination with carboplatin in patients who are not candidates for curative surgery or radiation therapy. The recommended dosing regimen for ABRAXANE for treatment of metastatic breast cancer is 260 mg/m² administered intravenously over 30 minutes every 3 weeks. The recommended dosage of ABRAXANE for treatment of NSCLC is 100 mg/m² administered as an intravenous infusion over 30 minutes on Days 1, 8, and 15 of each 21-day cycle. The recommended dose of carboplatin is AUC=6 mg*min/mL on Day 1 only of each 21-day cycle, beginning immediately after the completion of ABRAXANE administration.

Results from a clinical study involving 861 patients showed that 35% of people treated with a combination of ABRAXANE and gemcitabine were alive at the end of the first year as compared to only 22% who received gemcitabine only. Accordingly, in one embodiment, subjects diagnosed with pancreatic cancer, NSCLC or metastatic breast cancer, may be administered ABRAXANE as described above, in combination with intravenous infusion of KLTi at a dose of 20-50 g/day for a first time period of between 3-7 consecutive days, followed by a waiting period of between 1-5 days, which is then followed by administering a second dose of KLTi for a second time period of between 3-7 consecutive days, within a repeated 28-day cycle.

Mitomycin C has been shown to be useful in the therapy of disseminated adenocarcinoma of the stomach or pancreas in proven combinations with other approved chemotherapeutic agents and as palliative treatment when other modalities have failed. Mitomycin C is dosed at 15-20 mg/m² intravenously as a single dose via a functioning intravenous catheter at 6-8 week intervals. Accordingly, in one embodiment, subjects diagnosed with pancreatic cancer or metastatic breast cancer, may be administered mitomycin C at a dose stated above in combination with intravenous infusion of KLTi at a dose of 20-50 g/day for a first time period of between 3-7 consecutive days, followed by a waiting period of between 1-5 days, which is then followed by administering a second dose of KLTi for a second time period of between 3-7 consecutive days, within a repeated 28-day cycle.

### VIII. MEASURING THERAPEUTIC EFFICACY

Efficacy of the treatment may be monitored, for example, by CT scans of the patients to determine growth or progression of the tumor, or Karnofsky performance. The Karnofsky Performance Scale Index allows patients to be classified on a scale of 0 to 100 as to their functional impairment, with zero indicating death and 100 indicating no patient complaints and no evidence of disease. The primary efficacy outcome measure for the efficacy trial described in Example 2 was Progression Free Survival (PFS). PFS includes patient deaths and assumes that death is related to progression of cancer, a reasonable assumption with pancreatic cancer. PFS is defined as the time interval from randomization to objective tumor progression or death.

As shown in Example 2, efficacy of the treatment was evaluated in part using RECIST 1.1 RECIST refers to the Response Evaluation Criteria for Solid Tumors assessment tool an involves measuring tumors (lesions) in patient using, for example, CT or MRI. As noted above, RECIST Version 1.1 was utilized in evaluating patient responses. Complete response (CR) indicates that all target lesions are gone; partial response (PR) indicates that there was a ≧30% decrease from baseline; progressive disease (PD) indicates a ≧20% increase from the smallest sum of the longest diameter recorded since treatment started (best response); and stable disease (SD) indicates neither PD nor PR.

Treatment of a subject diagnosed with cancer using the methods described herein are considered to be effective, for example, if a the percent of subjects showing a complete or partial response (CR or PR) is greater for the group of subjects being administered the drug composition as compared to the group of subjects not being administered the drug composition or being in a control group. Treatment is considered effective if the percent of subjects in the group receiving KLTi in combination with a second drug (the experimental group) which show a CR or PR is significantly greater than the percent of subjects in the group receiving the second drug alone (the control group). For example, if the percent of subjects in the experimental group which exhibit CR or PR is at least 5, 10, 20, 30, or 50 percentage points greater than the percent of subjects in the control group, treatment of the experiment group is considered efficacious. Treatment efficacy as assessed using RECIST is also observed if the percent of subjects in the experimental group which exhibit stable disease (SD) or progressive disease (PD) is significantly less than the percent of subjects in the control group who exhibit SD or PD. For example, if the percent of subjects in the experimental group which exhibit SD or PD is at least 5, 10, 20, 30, or 50 percentage points less than the percent of subjects in the control group, treatment of the experiment group is considered efficacious. Such changes are measured at various points of time after the start of experimental treatment, for example, such changes may be measured at 1 month, 2 months, 3 months, 4 months, 5 months, 6 months, 7 months, 8 months, 9 months, 10 months, 11 months, 1 year, 16 months, 2 years, 30 months, 3 years, 4 years, and/or 5 years after the start of the treatment.

### EXAMPLES

The following examples are illustrative in nature and are in no way intended to be limiting.

### Example 1

### Preparation of Kanglaite Injection

The Coix seed oil emulsion as used herein was prepared by the Zhejiang Kanglaite Pharmaceutical Co., using seeds harvested from specified fields in the Zhejiang Province of China. No chemical fertilizers or pesticides were used in raising the crops, and plantings and harvestings were conducted at pre-determined times. The water-based emulsion was prepared to contain 10 g Coix seed oil per 100 ml of emulsion, with soybean phospholipid (1.5 g) and glycerin (2.5 g) added as emulsifiers. All batches were analyzed by high performance liquid chromatography (HPLC) using an Agilent 1100 HPLC with quaternary pump and an Alltech 500 ELSD detector. The temperature of drift tube was 70° C., with air at a flow rate of 1.2 L/minute used as carrier gas in a Zorbax extend C18 column with 5-μm particle size, 4.6 mm internal diameter and 250 mm length (Agilent Technologies, Santa Clara, Calif.) at 35° C., acetonitrile-dichlormethane (65:35) as mobile phase, at a 0.5 ml/min flow rate. Integration on each test result was conducted with the slope rate of 60 in the Agilent HP ChemStations system.

The same product marketed in China was used in the clinical trials described below. Following pooling studies for stability and extractables, a protocol was written directing participating pharmacies to pool the contents of the 100 mL bottles into 1000 mL ethylvinyl acetate bags ordinarily used for total parenteral nutrition (TPN). The TPN bags, containing 300 mL of KLTi were routinely transferred by courier to patient's homes, and stored there at room temperature awaiting use. The product was provided in the same 100 mL bottle which is sold in China. For the study described in Example 2, 3 one hundred mL bottles of KLTi were pooled under aseptic conditions into an ethyl vinyl acetate compounding TPN compounding bag provided for this purposed.

### Example 2

### Phase 2b Study of Kanglaite Injection Plus Gemcitabine with Advanced Pancreatic Cancer

A phase 2b, multi-center, randomized, open-label clinical trial compared the effect of gemcitabine in combination with KLTi to gemcitabine alone in patients with locally advanced or metastatic pancreatic cancer, not amenable to curative surgical resection.

A total of 41 patients were randomized to Cohort 1 with 38 included in the safety population, specifying all patients who received at least one dose of study drug. Thirty-seven of the 38 patients had been diagnosed as having Stage IV pancreatic cancer. Twenty-six patients were assigned to Group 1 to receive both KLTi and gemcitabine while 12 patients were assigned to Group 2 to receive only gemcitabine.

All patients received marketed gemcitabine, which was administered at 1000 mg/m² over a minimum of 30 min and a maximum of 38 min once weekly for 3 weeks, followed by a rest period of 13 days before the next gemcitabine dose. Based on a chemotherapy cycle of 28 days (4 weeks), gemcitabine was given on Days 1, 8, and 15 of the 28-day cycle. For patients in Group 1, KLTi infusion was administered on each of Days 1-5 for each of those same 3 weeks (i.e., Days 1-5, Days 8-12 and Days 15-19). Patients were then rested for the remaining 9 days of the cycle (Days 20-28).

Dosing of Group 1 patients included administration of KLTi at a dose of 30 g/day provided as a 300 mL volume. For the first 2 KLTi infusions for each patient, the infusion rate was started at 1.0 mL/min for the first 15 min, and then increased to 150 mL/hr until the KLTi was completely infused if no safety issues were apparent. Once safety was established in the first 2 infusions, all subsequent KLTi infusions were given at a target rate of 150 m L/hr. Beginning with cycle 2, the target rate of infusion may, however, have been increased to 200 mg/hr. The total infusion time for 300 mL of KLTi was therefore expected to be approximately 2 hr for Cycle 1 and approximately 90 min for all remaining cycles.

Duration of exposure to the test drug(s) differed between groups. Patients in Group 1 received more exposure to gemcitabine than did the patients in Group 2, as shown in Table 1 below.

TABLE 1

| | Group 1 | Group 2 |
|---|---|---|
| | (KLTi + Gemcidabine) | (Gemcidabine) |
| No. Cycles | | |
| Mean | 4.3 | 2.8 |
| Median | 4.0 | 2.0 |
| Duration of | | |
| Exposure (Days) | | |
| Mean | 111.6 KLTi/110 | 67.7 |
| | gemcitabin | |
| Median | 95.5 KLTi/90 | 43.0 |
| | gemcitabine | |

Progression Free Survival (PFS) was calculated from the date of randomization to the date of objective disease progression or death. Patients without objective disease progression or death were censored at their last evaluable tumor assessment. Table 2 below provides PFS data and shows that subjects in Group 1 experienced an approximately two-fold increase in the median PFS and that the rate of PFS was consistently greater for subjects in Group 1 as compared to Group 2 when measured at 1 month, 3 months, 4 months, 6 months and 1 year. This significant increase in PFS in Group 1 as compared to Group 2 is indicative of the significant advantage of administering KLTi in combination to gemcidabine as compared to administering gemcidabine alone to treat pancreatic cancer.

TABLE 2

| | Group 1 | Group 2 |
|---|---|---|
| | (KLTi + Gemcidabine) | (Gemcidabine) |
| Treatment (n) | 28 | 13 |
| Median PFS | 114 days | 57.5 days |
| N % Censored | 16 (57.1%) | 2 (15.4%) |
| PFS Rate | | |
| 1 month | 95.8% | 75% |
| 3 months | 78.2% | 25% |
| 4 months | 46.5% | 25% |
| 6 months | 34.9% | 8.3% |
| 1 year | 34.9% | 8.3% |

| | |
|---|---|
| P-value (log rank) | 0.0080 |
| Hazard Ratio | 0.338 |
| 95% Cl | (0.145, 0788) |

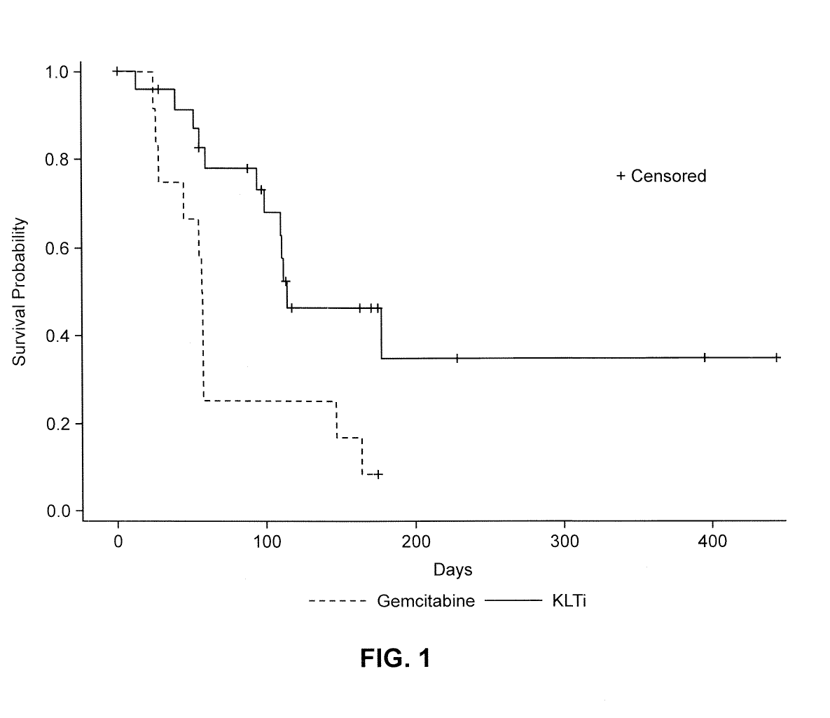

Secondary endpoints included overall response data using RECIST 1.1 criteria (Table 3) and overall survival (Table 4) were also assessed, as summarized below. As noted in this table, the overall response rate (ORR) was 14.3% for Group 1 patients, as compared to 7.7% for Group 2 patients. A graph depicting product-limit survival estimates based on the analysis of overall survival is shown in FIG. 1, comparing overall survival of patients in Group 1 vs. Group 2.

TABLE 3

| | Group 1 | Group 2 |
|---|---|---|
| | (KLTi + Gemcidabine) | (Gemcidabine) |
| Intent To Treat Population | | |
| CR + PR (%) | 4 (14.3%) | 1 (7.7%) |
| Complete Response (CR) | 0 (0)% | 0 (0%) |
| Partial Response (PR) | 4 (14.3%) | 1 (7.7%) |
| Stable Disease (SD) | 9 (32.1%) | 2 (15.4%) |
| Progressive Disease (PD) | 5 (17.9%) | 6 (46.2%) |
| Not Evaluable | 10 (35.7%) | 4 (30.8%) |
| Efficacy Evaluable | | |
| Population | | |
| CR + PR (%) | 4 (20.0%) | 1 (11.1%) |
| Complete Response (CR) | 0 (0)% | 0 (0%) |
| Partial Response (PR) | 4 (20.0%) | 1 (11.1%) |
| Stable Disease (SD) | 9 (45.0%) | 2 (22.2%) |
| Progressive Disease (PD) | 5 (25.0%) | 6 (66.7%) |
| Not Evaluable | 2 (10.0%) | 0 |

TABLE 4

| | Group 1 | Group 2 |
|---|---|---|
| | (KLTi + Gemcidabine) | (Gemcidabine) |
| Number of Patients | 28 | 13 |
| Survival/Median | 218 days (7.3 months) | 162 days (5.4 months) |

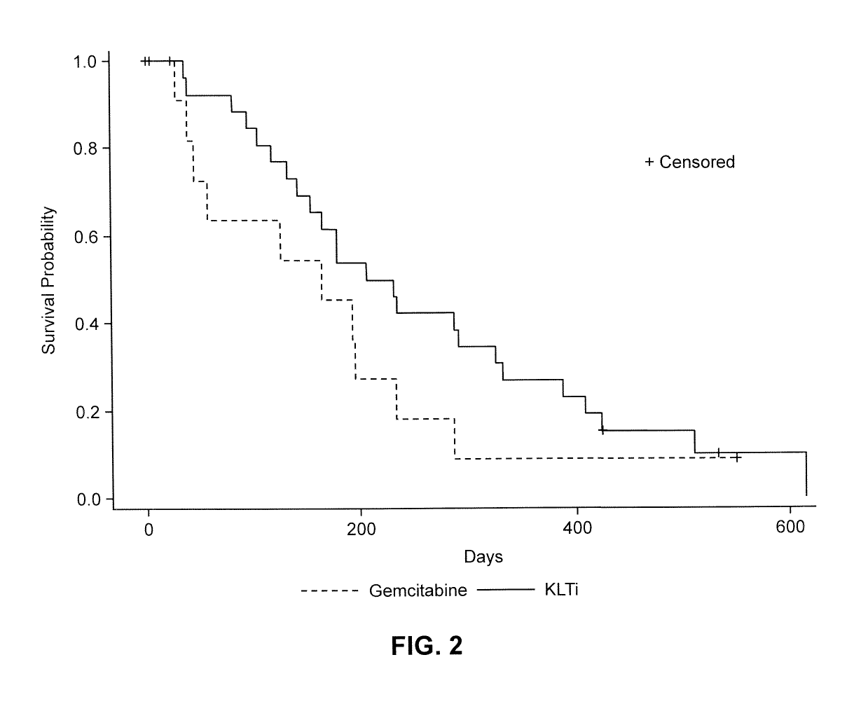

PFS between the Group 1 and Group 2 arms appears to be related to duration on treatment. The difference observed in product-limit survival estimates based on analysis of PFS for patients in Groups 1 and 2 is illustrated in FIG. 2.

With respect to safety analysis, more than 1900 infusions of KLTi were included in the analysis. No serious adverse events thought to be related to the study drug were observed, except for 1 patient who experienced transient confusion that completely resolved. Adverse events were comparable in the Group 1 and Group 2 arms of the study.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A treatment method, comprising:
administering to a subject intravenously a first dose of KLTi each day for a first dosing time period of 3 to 7 consecutive days.
waiting for a first resting time period of 1 to 5 days wherein during the resting time period KLTi is not administered, and
repeating the administering at least once at a second dose for a second dosing time period of 3 to 7 consecutive days,
wherein the subject has been diagnosed with a cancer.

2. The method of claim 1, wherein the first dose of KLTi comprises about 20 g/day to 50 g/day coix seed oil.

3. The method of claim 1, wherein the first dosing time period is 4 to 5 consecutive days.

4. The method of claim 1, wherein the first resting time period is 2 to 4 consecutive days.

5. The method of claim 1, wherein the second dose of KLTi comprises about 20 g/day to 50 g/day coix seed oil.

6. The method of claim 1, wherein the second dosing time period is 4 to 5 consecutive days.

7. The method of claim 1, further comprising waiting for a second resting time period of 1 to 5 days, wherein during the second resting time period KLTi is not administered.

8. The method of claim 7, further comprising administering a third dose of KLTi for a third dosing time period of 3 to 7 days consecutive days.

9. The method of claim 8, wherein the third dose of KLTi comprises about 20 g/day to 50 g/day coix seed oil.

10. The method of claim 8, further comprising waiting for a third resting period of 3 to 11 consecutive days.

11. The method of claim 1, wherein the cancer is selected from the group consisting of pancreatic, prostate, non small cell lung carcinoma, gastric, breast or liver cancer.

12. The method of claim 1, wherein the subject is being treating with a chemotherapeutic agent.

13. The method of claim 1, wherein the subject is being treated with a chemotherapeutic agent selected from the group consisting of fluorouracil, erlotinib hydrochloride, gemcitabine hydrochloride, mitomycin-C, ABRAXANE and FOLFIRINOX.

14-23. (canceled)
